(19) United States
(12) Patent Application Publication
(10) Pub. No.: US 2018/0315492 A1
(43) Pub. Date: Nov. 1, 2018

(54) COMMUNICATION DEVICES AND SYSTEMS AND METHODS OF ANALYZING, AUTHENTICATING, AND TRANSMITTING MEDICAL INFORMATION

(71) Applicant: Darroch Medical Solutions, Inc., San Diego, CA (US)

(72) Inventors: Colby Bishop, San Diego, CA (US); Anthony Shao, Hillsborough, CA (US); Jacques Yeager, Riverside, CA (US)

(73) Assignee: Darroch Medical Solutions, Inc., San Diego, CA (US)

(21) Appl. No.: 15/962,290

(22) Filed: Apr. 25, 2018

Related U.S. Application Data
(60) Provisional application No. 62/490,189, filed on Apr. 26, 2017.

(51) Int. Cl. G16H 10/60; H04L 29/06

(52) U.S. Cl. CPC G16H 10/60; H04L 63/08

(57) ABSTRACT

Communication devices and systems and methods of analyzing, authenticating, and transmitting medical information are provided herein. A communication device includes a housing, a transceiver circuit, a processor, and a battery circuit. The transceiver circuit has a communication channel. The processor is in communication with the transceiver circuit. The communication channel receives medical data from at least one medical instrument. The transceiver circuit transmits the medical data to a network or server. Disclosed systems and methods collect, analyze and build a proxy of patient health and response to treatment, securely authenticate, encrypt, and transmit data medical instruments to nurses and the EMR for use by medical professionals.

### CROSS-REFERENCE TO RELATED APPLICATION

This application claims priority to and benefit of U.S. Patent Application No. 62/490,189, filed Apr. 26, 2017, which is hereby incorporated by reference in its entirety.

### FIELD OF THE DISCLOSURE

The present disclosure relates to communication devices and systems and methods of analyzing, authenticating, and transmitting medical information.

### BACKGROUND

It is well known throughout the medical industry that hospitals are well behind the curve when it comes to modernization. Despite the recent advances in technology, hospitals still utilize various technologies and methods that seem to be far behind the modern era. Despite attempts to bring new solutions to Intensive Care Units (ICUs) and floor bed areas, many wards still use manual data entry and manpower as their preferred method of inputting data and assessing the health of their patients. In effect, medical practitioners still employ manual methods of entering data and taking notes. As a result, medical practitioners spend less time with their patients, which in turn, leads to more medical errors. Moreover, healthcare practitioners often miscommunicate, leading to more administrative work and the potential for error.

A patient's health status and physiological responses to treatment are monitored primarily by nurses, who expend much of their time (i.e., about 60%) on administrative work, leaving less time for patient interaction. In non-critical hospital units, this patient monitoring involves a significant amount of the nurse's time and effort with very little automation. A patient's health status and physiological responses to treatment are recorded by nurses as they attend to patients either during their rounds or in response to alarms, and then requires the nurse to transcribe relevant data to the electronic medical record (EMR) using the fixed network in the hospital, after which it is available to other healthcare staff who are providing care to the patient. Medical devices (such as Infusion Pumps take pertinent patient readings and measurements; this information is sent over a local area network (LAN) to an on-site server. The server, while controlling most of the software related to medical devices sends the data over Wi-Fi to various electronic medical record systems (EMRs).

In non-ICU wards, healthcare professionals must manually record information from a variety of medical instruments, usually on their clothing, hands, or whatever else is available. This quick writing often introduces the possibility for transcription errors which can ripple down the hospital chain and cause medical errors. Moreover, nurses are often overworked while hospitals are understaffed, and this causes nurses to waste time in collecting information and obtaining proper medication for their patients

These procedures are a significant administrative burden on nurses, reduce their ability to attend to patients, and is a source of medical errors from transcription errors or delays in availability of patient data to other healthcare providers. While the data in EMR is accessible from a nurse station, this data is commonly not available to nurses in real time if they are not at the patient's bedside.

Currently, medical instruments transfer data via the hospital's Wi-Fi infrastructure using vendor supplied closed proprietary networks. Data transfer to the hospital's EMR often requires dedicated servers installed and maintained by vendors. These servers also control most of the software related to medical instruments. Security protocols used between the server and instruments often involve unsecured communication once the initial authentication process is complete. As a result, both the hospital Wi-Fi infrastructure and the communication networks used for data transfer from instrument to the EMR are vulnerable to various cybersecurity attacks and denial of services.

From another perspective, hospitals are also easily susceptible to cyber-security attacks in the form of “denial-of-service” attacks (DOS). These kinds of attacks can prevent medical instruments from working, thus either injuring or killing a patient. A DOS attack, also known as ransomware, is when a third party intercepts the line of communication between a device and its associated server. Once a third party has access to this line, he or she can inundate the device with empty packets of data; since the device is unable to distinguish between relevant and irrelevant packets, the device attempts to read each, eventually causing the system to over work or shut down. In turn, this can either fatally injure or kill a patient. Worse, there are many more kinds of cybersecurity attacks that hospitals are facing every day. The hospital needs some sort of device that can act as a “security middleman” and prevent instrumentation from being shut down remotely. The need to improve patient care, increase efficiency, decrease errors, and maintain information integrity are always pertinent to the hospital environment.

Thus, there is a need for There is a need for a device, system and method that can coordinate, analyze, and present data in a cohesive manner would greatly increase the quality of care in ICU and non-ICU wards in hospitals. There is a need for a device, system and method that can collect medical data from medical instruments and provide provides a continuous running view of patient health and response to treatment to medical practitioners anywhere they go as they move about a hospital. There is also a need for a device, system and method which can act as a patient proxy between various medical instruments and medical practitioners.

### SUMMARY

The present disclosure, in its many embodiments, alleviates to a great extent the disadvantages of hospital medical data solutions. The present disclosure comprises a device that is capable of a direct secondary means of communication between nurses and the medical instruments used to monitor the hospitalized patient's health status and physiological responses to treatment. It collects and analyzes data from medical instruments, develops a “proxy” of the patient's health status and responses to treatment, and presents a proxy to nurses on mobile and fixed devices. The device provides higher levels of security by using secure communication with instruments and nurses, and by combining Wi-Fi with short range technology options such as Bluetooth and/or ZigBee.

Disclosed embodiments have the potential to improve efficiency and effectiveness of nurses and reduce the number of medical errors, complications, or deaths during treatment. Embodiments will securitize the flow of highly sensitive patient health information and reduce the possibility for nefarious or unauthorized access of this data. Embodiments will contribute to an overall increase in the efficiency of healthcare provided throughout the United States.

Disclosed embodiments comprise hardware, software and firmware designed to collect, coordinate, analyze, and secure data from various bedside medical instruments and accessories, and provide a continuous running view of patient health and response to treatment. Exemplary embodiments aim to increase the quality and efficiency of medical care and reduce medical errors while decreasing security incidents and information theft that currently plague medical information systems.

Objects of the present disclosure include providing electronic transfer of data, as opposed to manual input; easy connectability with medical devices commonly used during treatment in hospitals; installation with minimal user input with exception of turning it on/plugging it in; a full system representing a cost effective solution; limiting the size of the device to ensure maximum portability (e.g., under 6″×6″×4″, and under 2 lbs.); protect data transfer from security threats such as man in the middle attacks, especially those involving denial of service.

In exemplary embodiments, the communication device has the following features and capabilities: a failure rate of at most 1 device failure per 10⁶ hours of operation; the ability to receive data from up to 7 medical instruments; the ability to pulse information to the hospital's network or a cloud database; small enough size to be hidden from view; and the ability to move freely with the patients, including having a battery charging system to address the issue of portability, the ability to hold a charge for at least an hour, and the ability to easily to sanitize it as it moves throughout the hospital.

Exemplary embodiments reduce the time spent on administrative tasks by providing a new device called “The POD.” The POD is a small, portable device capable of being moved with patients from room-to-room and ward-to-ward. Functionally, the POD acts as a patient proxy between various medical instruments and medical practitioners. Via Bluetooth and Wi-Fi connection, the POD is able to pair and pull data from various instruments, coordinate the data, and present it in a cohesive, real-time manner to medical practitioners. The POD thus has the ability to help reduce the amount of raw data practitioners need to view and record; moreover, the coordination of this data can aid in making the best decision.

Exemplary embodiments may use Wi-Fi, Bluetooth, ZigBee, and/or other wireless communication methods to collect, analyze and build a proxy of patient health and response to treatment, securely authenticate, encrypt, and transmit data medical instruments to nurses and the EMR for use by medical professionals. Exemplary embodiments can be implemented in a variety of medical spaces, such as community hospitals, and interface with a wide variety of medical instruments. Exemplary devices are small enough to be hidden out of sight, an added precaution from local attacks, and it is mobile enough to move with patients from various units or healthcare facilities.

Exemplary embodiments of a POD, or communication device, comprise a housing, a at least one transceiver circuit disposed within the housing, a processor in communication with the transceiver circuit, and a battery circuit disposed within the housing. The transceiver circuit has at a communication channel. The communication channel receives medical data from at least one medical instrument. In exemplary embodiments, the transceiver circuit transmits the medical data to a network or server.

In exemplary embodiments, the at least one medical instrument is located in a room and transceiver circuit does not receive local medical data when located outside the room. The processor may develop a health status proxy based upon the medical data. The medical data may be received, authenticated, and transmitted in real time. In exemplary embodiments, the network or server contains a patient data hub and the first or second transceiver transmits a key to the patient data hub.

The communication device may further comprise a user interface displaying medical information of a patient including, but not limited to, one or more of: the patient's temperature, the patient's pulse, the patient's PO2, the patient's respiration rate, and a volume of fluid infused into the patient. The user interface may enable a medical practitioner to set medical information parameters such that the communication device will monitor for deviations from the set medical information parameters. In exemplary embodiments, the communication device further comprises a warning system alerting a medical practitioner that a patient requires medical attention.

In exemplary embodiments, a computer-implemented system of analyzing, authenticating, and transmitting medical information, comprises one or more medical instruments, a server or network, and a communication device in communication with the one or more medical instruments and the server or network. the communication device includes a housing, a transceiver circuit disposed within the housing, a processor in communication with the transceiver circuit, and a battery circuit disposed within the housing. The transceiver circuit has at a communication channel. The communication channel receives medical data from at least one medical instrument. In exemplary embodiments, the transceiver circuit transmits the medical data to a network or server.

In exemplary embodiments, the network or server comprises a port that automatically opens such that the network or server listens for and connects to a first medical instrument of the one or more medical instruments. After the network or server completes a connection to the first medical instrument, the network or server may automatically listen for and connects to a second medical instrument. The network or server may contain a patient data hub and the first or second transceiver transmits a key to the patient data hub. In exemplary embodiments, the one or more medical instruments comprise a plurality of medical instruments connected to a single patient. The system may further comprise a warning system alerting a medical practitioner that a patient requires medical attention.

Exemplary computer-implemented methods of analyzing, authenticating, and transmitting medical information comprise receiving medical data from one or more medical instruments, authenticating the medical data, transmitting the medical data to a network or server, and developing a health status proxy based upon the medical data. The receiving, authenticating, and transmitting steps are performed in real time. The network or server may contain a patient data hub and may further comprise transmitting a key to the patient data hub. Exemplary methods further comprise automatically listening for and connecting to a communication device that performs the receiving, authenticating, and transmitting steps. Exemplary methods further comprise automatically listening for and connecting to a first medical instrument of the one or more medical instruments. Exemplary methods further comprise alerting a medical practitioner that a patient requires medical attention.

Accordingly, it is seen that devices, systems, and methods of analyzing, authenticating, and transmitting medical information are provided. These and other features and advantages will be appreciated from review of the following detailed description, along with the accompanying figures in which like reference numbers refer to like parts throughout.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a perspective view of an exemplary embodiment of a communication device in accordance with the present disclosure;

FIG. 2 is a schematic of an exemplary embodiment of a system of analyzing, authenticating, and transmitting medical information in accordance with the present disclosure;

FIG. 3 is a perspective view of an exemplary embodiment of a housing for a communication device in accordance with the present disclosure;

FIG. 4 is a perspective view of an exemplary embodiment of a battery charging circuit in accordance with the present disclosure;

FIG. 5 is a perspective view of an exemplary embodiment of a battery charging circuit in accordance with the present disclosure;

FIG. 6 is a schematic of an exemplary embodiment of a battery charging circuit in accordance with the present disclosure;

FIG. 7 is a schematic of an exemplary embodiment of a system and method of analyzing, authenticating, and transmitting medical information in accordance with the present disclosure;

FIG. 8 is a schematic of an exemplary embodiment of an encrypted communications subsystem in accordance with the present disclosure;

FIG. 9 is an illustration of an exemplary embodiment of a user interface in accordance with the present disclosure;

FIG. 10A is an illustration of an exemplary embodiment of a user interface in accordance with the present disclosure;

FIG. 10B is an illustration of an exemplary embodiment of a user interface in accordance with the present disclosure; and

FIG. 11 is a schematic of an exemplary embodiment of a system and method of analyzing, authenticating, and transmitting medical information in accordance with the present disclosure.

### DETAILED DESCRIPTION

In the following detailed description of exemplary embodiments of the disclosure, reference is made to the accompanying drawings in which like references indicate similar elements, and in which is shown by way of illustration specific embodiments in which disclosed systems and devices may be practiced. These embodiments are described in sufficient detail to enable those skilled in the art to practice the embodiments, and it is to be understood that other embodiments may be utilized and that logical, mechanical, functional, and other changes may be made without departing from the scope of the present disclosure. The following detailed description is, therefore, not to be taken in a limiting sense, and the scope of the present disclosure is defined by the appended claims. As used in the present disclosure, the term “or” shall be understood to be defined as a logical disjunction and shall not indicate an exclusive disjunction.

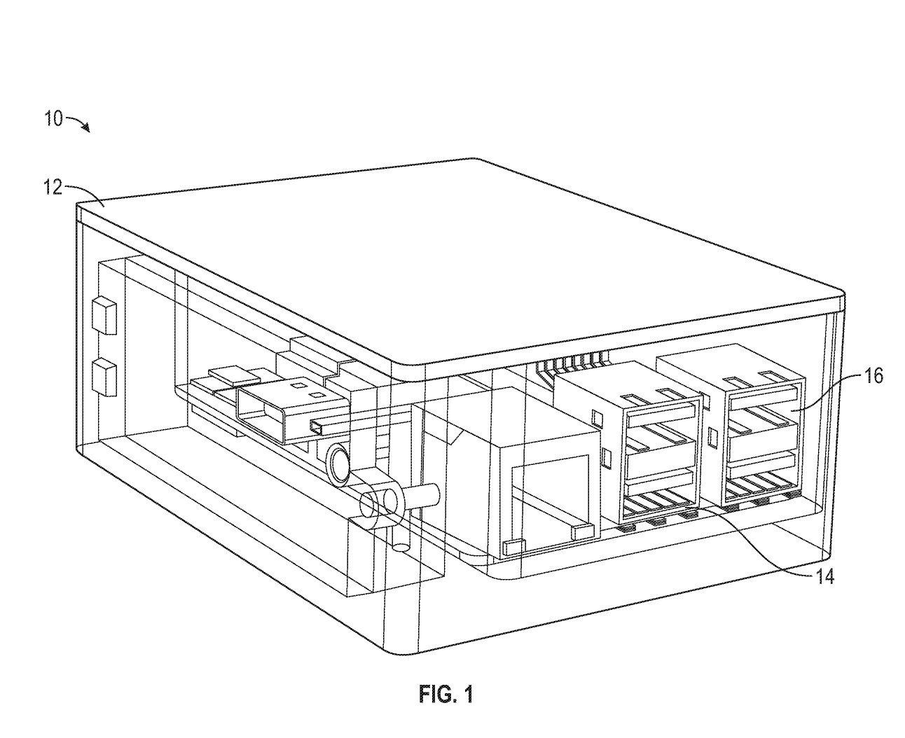

FIG. 1 illustrates an exemplary embodiment of a communication device 10, which has a housing 12 and at least one transceiver and transceiver circuit 14 in the housing. The main functions of the device are to receive and coordinate data from various medical instruments and present it in a cohesive manner to any medical practitioner qualified to view it. The device 10 contains a combination of hardware, software and firmware and may operate as a patient proxy, collecting, amalgamating and analyzing in real-time information from various medical instruments connected to the single patient it is configured to be paired.

Used as part of a system analyzing, authenticating, and transmitting medical information, the device 10 has the ability to communicate with nurses and the hospital's EMR connected to the hospital Wi-Fi or a secondary channel established by the device. In doing so, the device transforms disparate information into an integrated information system for gathering and disseminating time sensitive and critical health status and physiological responses to treatment from patients. The device, using some combination of hardware, software and firmware, receives information from a medical instrument or group of medical instruments, analyzes and develops a proxy of the patient's health status and response to treatment. Disclosed embodiments create a more efficient means of transferring data from medical devices to the health care practitioners as well as EMR systems.

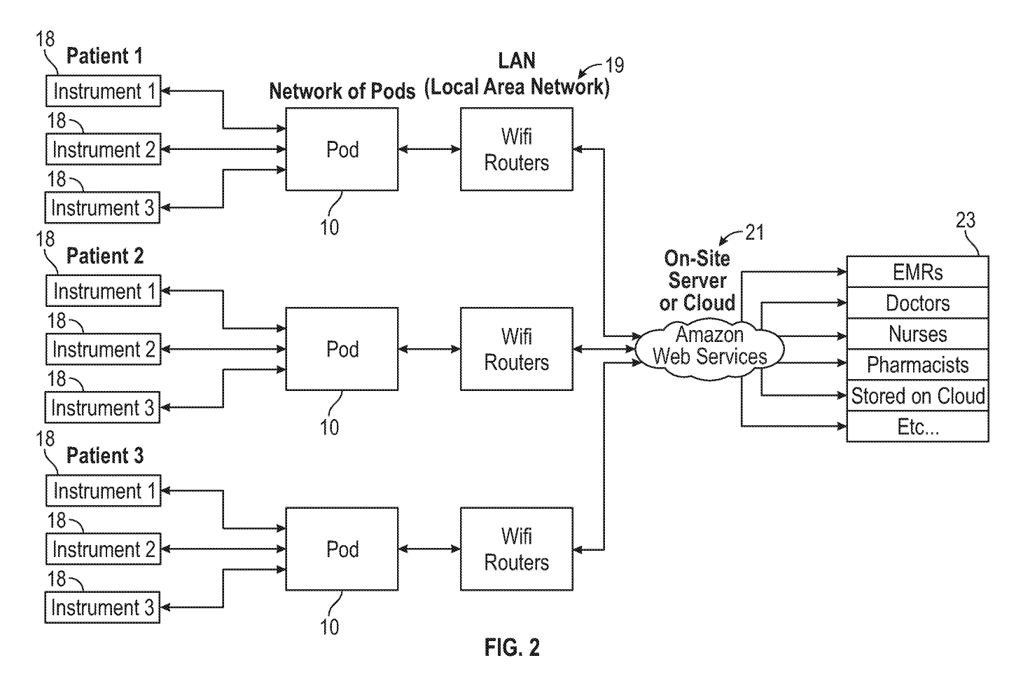

In exemplary embodiments, the communication device 10 has a first transceiver and circuit 14 having a communication channel. The device 10 has a processor (not shown) in communication with the transceiver circuit 14. Typically, embodiments will have one transceiver circuit including one transceiver, but some embodiments may have a second transceiver circuit 16. As shown in FIG. 2, the communication channel receives medical data from at least one medical instrument 18, which may be equipped with a transceiver or dongle. The medical instrument could be an infusion pump, or any other medical instrument that monitors or provides medication or treatment to patients. In the current hospital environment, medical devices (such as infusion pumps) take pertinent patient readings and measurements. This information is sent over a local area network (LAN) 19 to an on-site server 21. The server 21, while controlling most of the software related to medical devices sends the data over Wi-Fi to various electronic medical record systems (EMRs) 23. In exemplary embodiments, the communication device 10 transmits the medical data to the hospital's network or server. More particularly, and as described in detail herein, the communication device 10 is able to pull data from medical instruments, coordinate and analyze that information before sending it to its final destination.

Some infusion pumps monitor around a hundred variables (i.e. flow rate, time elapsed, drip count, etc.). Each variable you want to measure is recorded four times a second. They exist in the infusion pump as strings of numbers, on average five digits in length, and at four bits per number on the transfer, totaling ˜1.6 Kbps minimum data transfer rate. With multiple medical instruments 18 transmitting to the communication device 10, accounting for packet handling, a minimum of 7 Kbps is required for four connected medical instruments.

From a signal and communications perspective, exemplary embodiments communication device 10 and the medical instruments should operate only within the bounds of the room. In other words, in exemplary embodiments, the Bluetooth communication will be able to communicate and pair with the communication device 10 within a limited area; the signal must be weak enough to be undetectable outside a given area but strong enough to maintain a line of communication with the POD. In exemplary embodiments, at least one medical instrument 18 is located in a room and the transceiver circuit 14 does not receive medical data from the instrument 18 when located outside the room.

In exemplary embodiments, authentication and security features are provided. These may be provided via a second transceiver circuit. The second transceiver circuit 16 may be a low power circuit. In exemplary embodiments, at least one medical instrument 18 is located in a room and the second transceiver circuit 16 does not receive local authentication data when located outside the room. This is because the low-power transceiver 16 at the communication device 10 and medical devices 18 will ensure that only the devices in that room be able to see that signal. In other words, this prevents any sort of non-local attack on the medical instruments and communication device 10 because the authentication prevents any sort of man-in-the-middle attacks. In exemplary embodiments, Bluetooth communication does not extend past 10-25 ft. from the point source of the communication device 10. Also, the lower power draw will allow for a longer battery life while the communication device 10 is in transit with the patient.

It should be noted that the communication device 10 does not aim to replace any existing medical technologies; this would be too costly for most hospitals (especially community hospitals) to implement. Rather, the device 10 retroactively fits to existing technology is more appropriate for most hospital wards. Disclosed systems may implement one or more of the following codes and standards; C2-C2017—2017 National Electrical Safety Code® (NESC®), ASME Y14.5—2009, IEEE 81—2012, IEEE 1012—2016, ISO 13485:2016. In addition, the idea of using a less invasive approach would give medical practitioners and easier time adjusting to the POD. The removal of medical instruments on the network would free up a significant amount of bandwidth on the network itself. Exemplary embodiments use Wi-Fi as the main form of communication, as this is the communication standard used by medical instruments to communicate with the hospital's network and onsite servers. Using 802.11n, the communication device 10 can access both ranges and adapt to the hospital network and device manufacturers' preference.

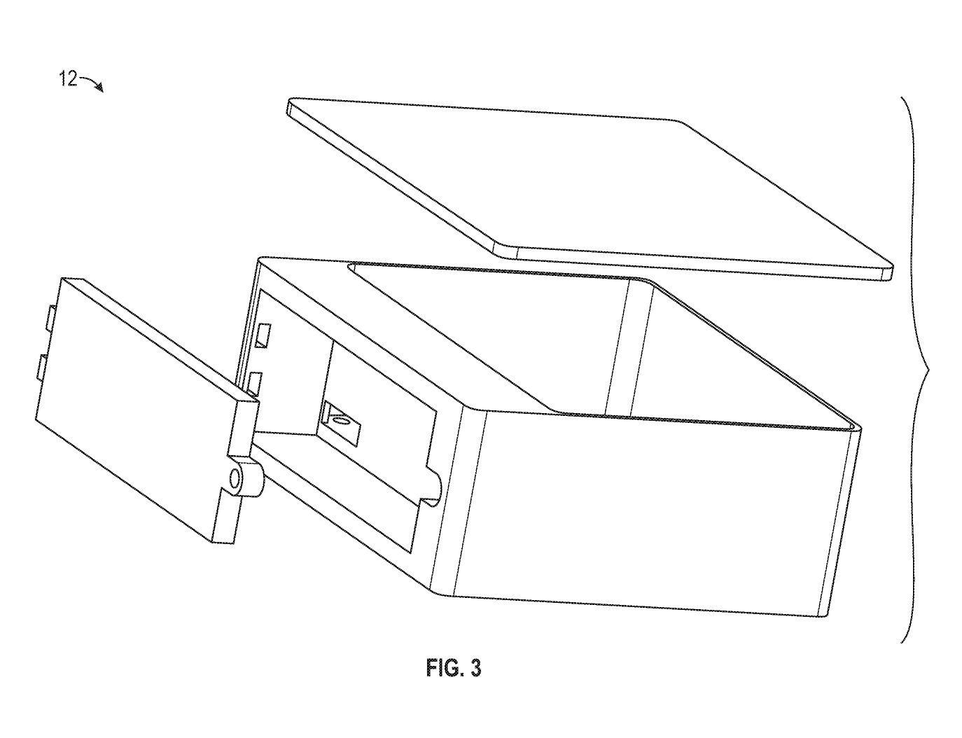

In exemplary embodiments, the housing 12, illustrated in FIG. 3, is made of materials designed to secure the circuitry inside and withstand moderate vibrations encountered. The device 10 may be sanitized to comply with the hospital environment it will be placed in. The device 10 is relatively small and so it is mobile enough to move with the patient to and from various units or healthcare facilities and can be stored or hidden out of sight, an added precaution from local attacks. In exemplary embodiments, the housing is a rectangular physical enclosure with dimensions of between about 1 inch×1 inch×0.2 inches and about 10 inches×18 inches×14 inches. In exemplary embodiments, the housing dimensions are about 6 inches×6 inches times 4 inches. In exemplary embodiments, the device 10 weighs up to about 10 pounds. Countermeasures such as a cooled enclosure, heatsinks, and fans may be designed to dissipate the heat generated from the device and will be implemented to reduce the risk of fire.

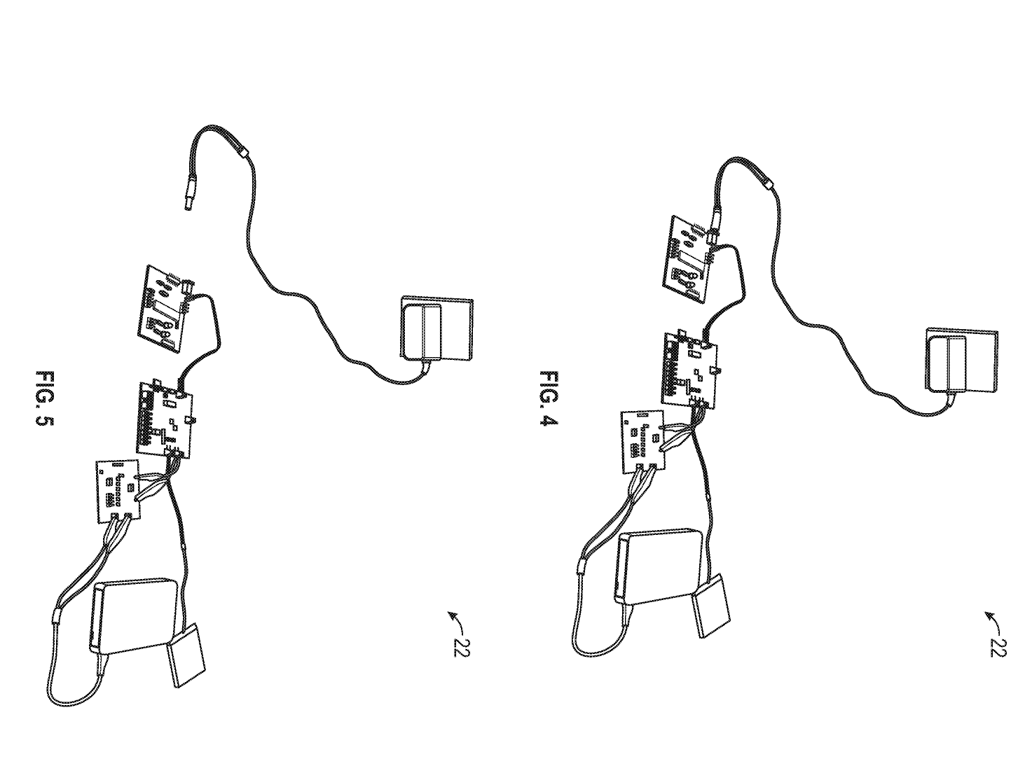

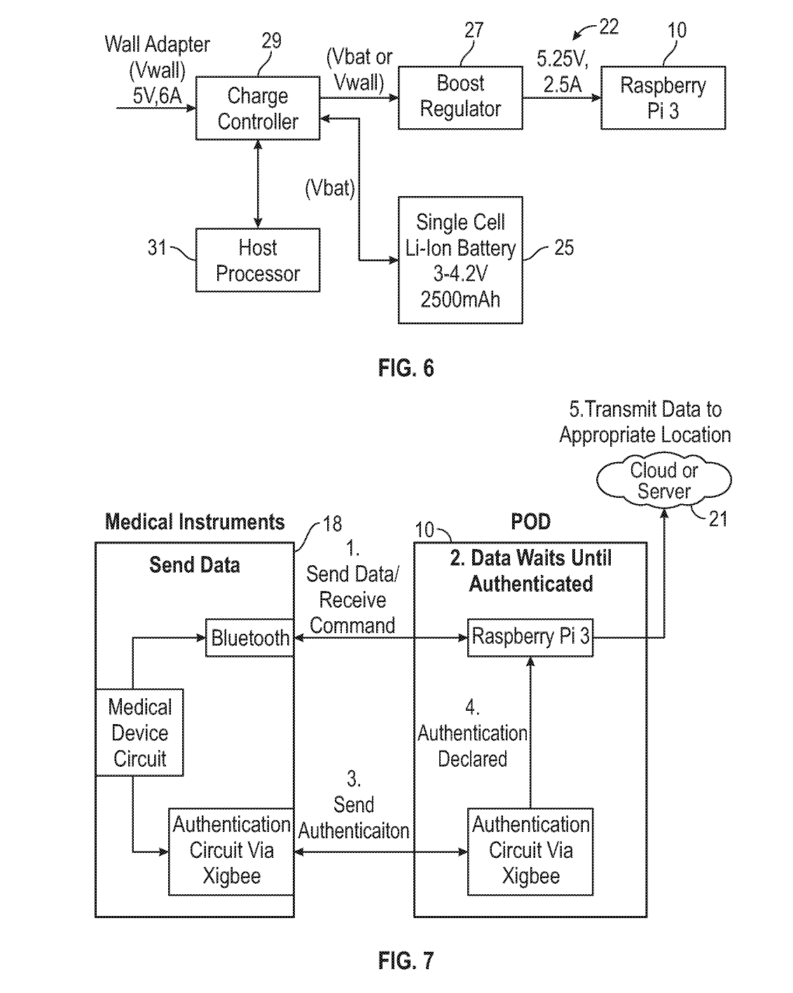

Turning to FIGS. 5A-6, in exemplary embodiments, a battery and battery circuit 22 are disposed within the housing 12 of the communication device 10. It will be able to power itself for at least two hours so that is able to move with patient. As shown in FIG. 6, in exemplary embodiments, this subsystem has at least one cell lithium-ion battery 25, a boost regulator 27 to bump the batteries output from 3V-4.2V to 5.25V, a charge controller 29 to safely charge the battery when the voltage gets too low, and a host processor 31 to monitor the set limits for the charge controller. The charge controller takes an input of 5V and up to 6 A and uses that to charge the battery while still keeping the communication device 10 running. It is itself controlled by a host processor that sets important parameters. The circuit could be a wall-powered charging circuit, illustrated in FIG. 4, or a battery charged charging circuit, shown in FIG. 5.

The battery circuit satisfies the need to move the communication device to and from different locations. If, for some reason, the patient is not in a room for a certain amount of time, the communication can hold a battery charge to continue its capabilities. In addition, this feature would also be of use in case of a power-outage or a lack of electricity. The battery circuit may have two modes of operation. One mode will draw power directly from the battery to power the system (not-charging), while the second mode of operation will draw power from the wall to power the system (charging).

The device's ability to receive the data is done in a secure manner, utilizing various channels of communication such as Bluetooth and Wi-Fi. The combination of several communication channels and a security circuit prevent local and nonlocal cybersecurity attacks. In exemplary embodiments, a second communication channel receives local authentication data verifying authenticity of the medical data. Side-band authentication is the second subsystem that concentrates upon the device's ability to conduct secure communications to and from each medical device. It is a means of securely transferring data using two different communication channels. It can act as a means of “local authentication” for the various devices, sending encrypted data to the device and then to the hospital network. The Bluetooth channel may be used to pair and authenticated devices to the communication device 10 and the Wi-Fi channel will be used to send and receive data to and from the medical instruments. In exemplary embodiments, the device operates a medical information platform that authenticates and transmits real-time medical information from multiple medical instruments, connected to a single patient, to nurses and EMR/EHR.

In exemplary embodiments, the second communication channel will be transferring less than one tenth of the data transferred on the main band, as it will consist of a checksum and other small amounts of conformational data. At less than 10 Kbps this will be fully realizable by the ZigBee channel, which has a max data rate of 250 Kbps, and can handle around ˜30 Kbps in actual usage, more than enough to send the desired 10 Kbps for the authentication band.

Securitization involves, but is not limited to, authentication and encryption. Encryption may be run on three distinct channels of communication: Wi-Fi, ZigBee and Bluetooth. In exemplary embodiments, the encryption is based on the Secure Shell network protocol and utilizes, but is not limited to, client-server architectures as the main method of encrypting data. The medical instrument acts as the client, and the device will act as the server. Once the server authenticates the instruments as clients on its sub-network, it calls for data from the instruments, and the instrument sends the data. The device then encrypts the data and send it forward to the nurse or the EMR/EHR. FIG. 7 shows a schematic of an exemplary authentication process. In exemplary embodiments, when a medical instrument 18 receives a command from the on-site server or transmits data to the EMR, it will require some form of authentication before completion. In essence, the communication device 10 will act as a “security node” that will authenticate and encrypt any and all data/commands being sent to and from the medical devices.

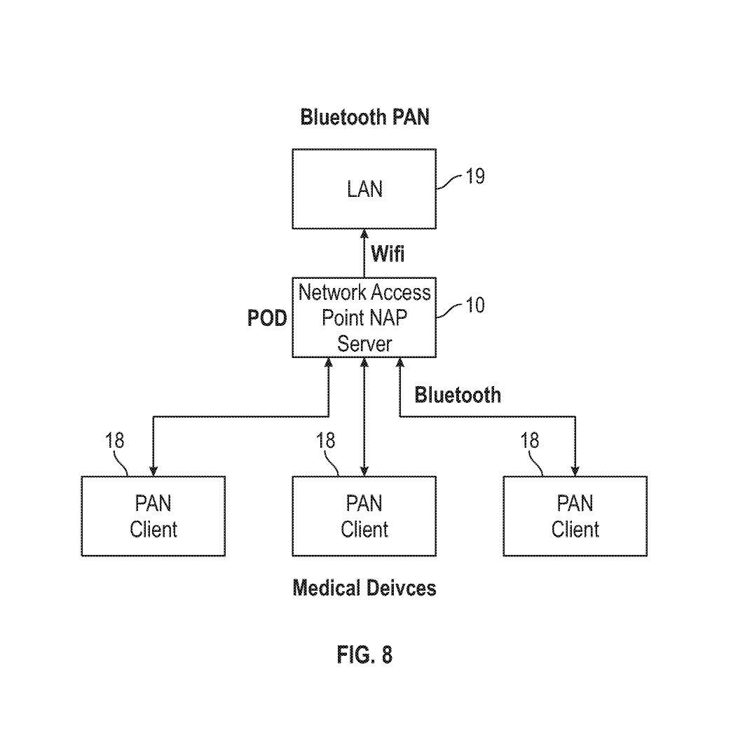

Referring to FIG. 8, in exemplary embodiments, the encrypted communications subsystem is split into the development of two different components: Encryption and Bluetooth PAN. The relationship between the Bluetooth channel and RP3 may be reconfigured to be a Bluetooth PAN (Personal-Area Network); the reason for this is because Bluetooth utilizes MAC addresses (48-bit address) to establish connections between devices. The Bluetooth PAN will configure the communication device 10 to act as a NAP or Network Access Point, similar to a network router. Up to 7 Bluetooth channels can connect to this NAP at one time, and this allows the communication device 10 to communicate in this configuration with multiple Bluetooth channels simultaneously. FIG. 8 represents the basic layout of how the communication device 10 fits into the PAN.

In exemplary embodiments, the communication device 10 and system include early warning systems such as alerting the nurse when an IV bag needs to be changed and alerting the nurse when certain vitals are declining. Moreover, nurses and doctors have the option to set parameters that the communication device 10 will watch for. If certain data goes outside these parameters, such as a high temperature, the communication device 10 will give the nurse or doctor the option to log this number into the patient's health records.

In operation, exemplary systems and methods collect, analyze, authenticate, encrypt, and securely medical data and proxy to nurses and EMR/EHR via several subsystems: Communication, Microanalytics, and Securitization. Some embodiments may specifically comprise Wireless Communication (Wi-Fi, ZigBee, and Bluetooth), Bluetooth PAN, battery charging circuitry, side-band authentication channels, and microanalytics subsystems. In exemplary embodiments, each patient will have their own dedicated communication device 10 and will be able to communicate and connect to a variety of medical instruments, creating patient-specific environments for each individual patient. Patients can move from ward-to-ward or hospital-to-hospital with their device. In this way, doctors and nurses can see the same information at the same time, aiding in their ability to coordinate their care. Moreover, nurses will not need to manually transcribe information which will reduce the amount of medical errors related to information input.

FIG. 2 is a schematic which demonstrates how exemplary systems and methods work. Multiple medical instruments 18, once connected to the communication device 10 will be able to connect to the device to transmit information to the nurse and EMR/EHR. It should be noted that in the figures and explanations, the medical instrument known as the “infusion pump”, which delivers fluids. nutrients and medications into a patient's body in controlled amounts will be used for illustrative purposes, though the device is equally applicable to other widely used medical instruments. Once the data is generated from the medical instrument 18 and sent to the communication device 10, the device will then transmit that information via Wi-Fi to an on-site server or a cloud-based storage system (“server/cloud”) 21.

In exemplary embodiments, communication is capable of being run on three channels (Wi-Fi, ZigBee and Bluetooth). FIG. 7 shows it being run on Wi-Fi and ZigBee channels. The device can establish Peer-to-Peer (P2P), Peer-to-Multipeer (P2M) or broadcast connections over the channels. Once syncing is complete between the instruments around a patient and the device, data transfer is unidirectional from the instrument to the device, and bi-directional between the nurse or EMR/EHR and the device.

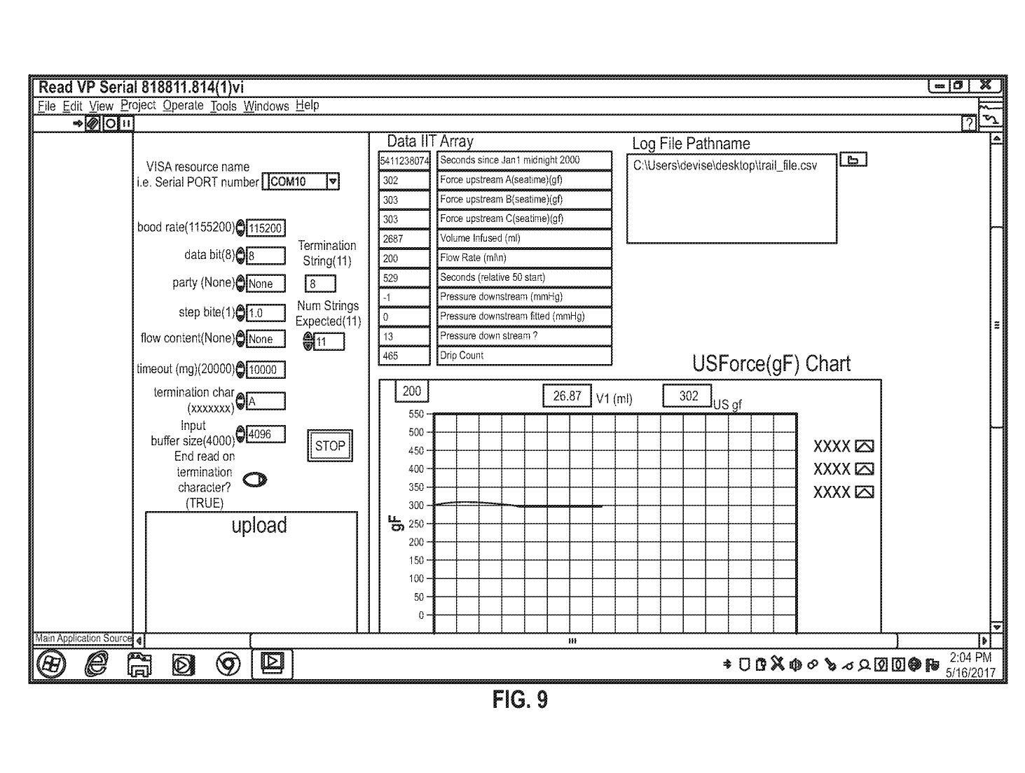

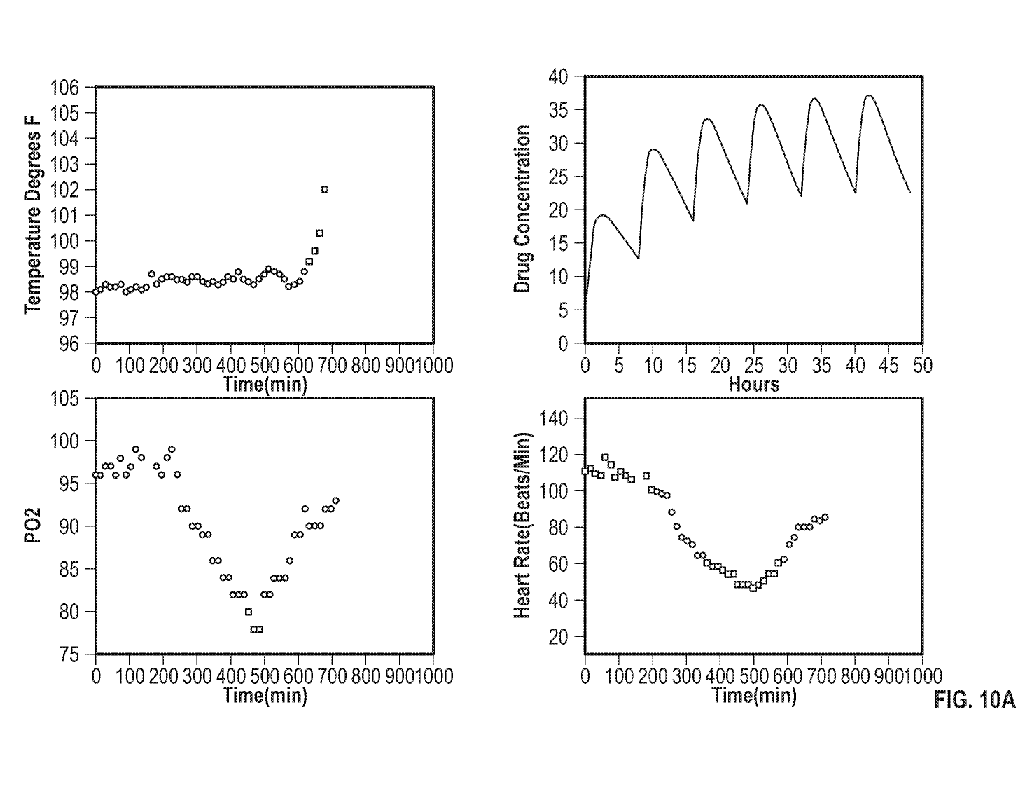

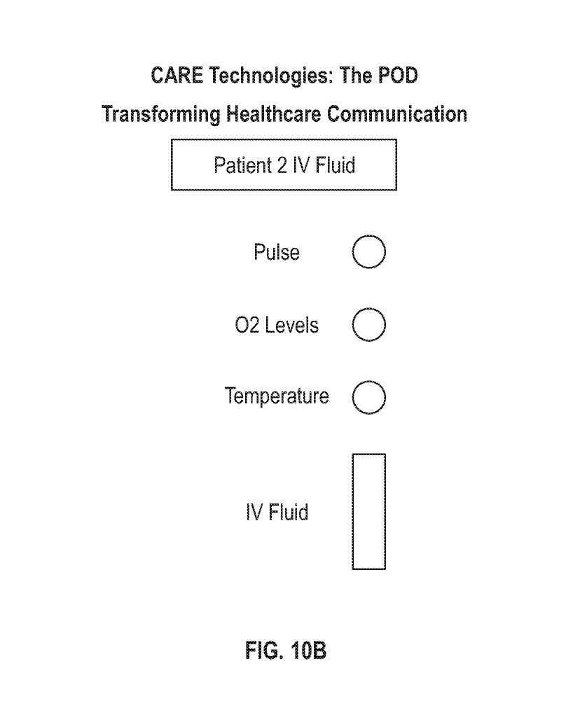

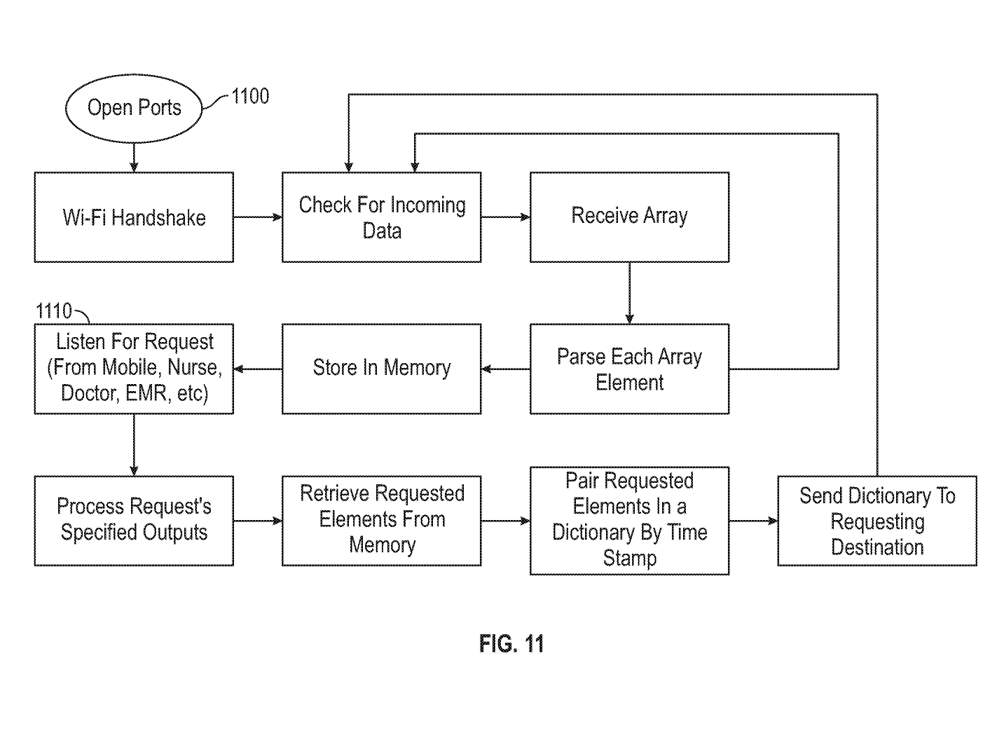

FIG. 2 shows an example of the data flow involved in the system. The communication device 10 takes medical data from various medical instruments 18 (infusion pumps, vital sign monitors, etc.) and coordinates, analyzes, and secures the data. This data flowing through the device 10 is then made readily available to healthcare practitioners around the hospital by placing the information on a cloud-based database or on the hospital's Wi-Fi through a local area network (LAN). The communication device 10 successfully displays the status of a patient's temperature, pulse, PO2, respiration rate, and amount of fluid infused into the body in real time. An exemplary user interface is shown in FIG. 9. If a problem arises (such as Patient 2's IV fluid), the nurse will be able to expand the information provided by clicking on the patient. By clicking on Patient 1, the nurse can see all the relevant vitals provided in real time. FIG. 10A shows this expanded view and FIG. 10B shows further information made available on the user interface. The user interface may be a mobile user interface, i.e., a display on a mobile device such as a smartphone or tablet.

Referring now to FIG. 11, in exemplary embodiments, the device or client can automatically enter into “searching mode” while looking for a device/server to connect to. Exemplary systems and methods can use the Bluetooth channel to establish the connection over a desired port; the client will continuously attempt to connect until the server is found and a connection is established. On the server side of the communications, the system automatically opens a port 1100, listens for a request 1110, and then connects to a device. Once the connection is closed, the server automatically begins listening and waiting to accept another device. In this way, multiple communication devices are directly connected to the cloud or hospital server.

Bluetooth has a range of ˜30 ft, operating at a data rate of up to 25 Mbps, and a low power draw of ˜100 mA average through transmit/receive cycles. Exemplary embodiments include a PAN Bluetooth network between multiple communication devices (in lieu of medical instruments temporarily). Wi-Fi operates on the 2.4 GHz and 5 GHz bands, with data rates up to 250 Mbps, well over the needs of medical data transfer. Wi-Fi uses ˜200 mA more to transmit and receive the same amount of data as Bluetooth. Utilizing arch Linux on the Raspberry Pi 3, exemplary communication devices operate in part as a virtual access point (AP), allowing medical devices to connect directly to the communication device over Wi-Fi and transfer data to be analyzed, displayed and/or transferred on to the EMR and onsite servers. In some embodiments, ZigBee could be the third mode of wireless communication implemented. XBEE Series 1 can be used for basic serial transfer from device to device. This acts as the second channel of the POD providing the side-band authentication security discussed herein.

In exemplary embodiments, microanalytics performed on the communication device 10 develops a proxy of the patient's health status and response to treatment. In exemplary embodiments, the processor develops a health status proxy based upon the medical data. The device 10 collects information from instruments such as, but not limited to, vital sign monitors, infusion pumps and peripheral devices that involved in the treatment of the patient. A communication device 10 will be given to each patient and can implement analytics in order to show data in real time. Analytics include, but are not limited to, removal of data artifacts, data trends on individual data channels, and cross-channel data analysis. The microanalytics subsystem will reduce the amount of time nurses and doctors will spend manually inputting data into their systems and will allow the nurse to better manage her time, allowing more time to spend actually caring for the patient.

The microanalytics subsystem may use numerical computing software to conduct various microanalytics on the data surrounding a patient. Although such software can simulate the analysis of data in real time, it can neither analyze the data in real time nor create a functioning user interface; therefore, future programs that will do analysis on the data in real time may be written in Python or other suitable languages. For the microanalytics to be useful, the analytics are displayed on a user-friendly interface. This UI compliments the data so that it provides the most usefulness to the medical practitioners using it.

In exemplary embodiments, the communication device 10 and system include early warning systems such as alerting the nurse when an IV bag needs to be changed and alerting the nurse when certain vitals are declining. Moreover, nurses and doctors have the option to set parameters that the communication device 10 will watch for. If certain data goes outside these parameters, such as a high temperature, the communication device 10 will give the nurse or doctor the option to log this number into the patient's health records.

From a security aspect, exemplary systems and methods create an added layer of security to prevent any form of cybersecurity attacks. Functionally, they can create this via the inherent security protocol of the Bluetooth communication or the “side-band” approach. Once a medical device has relevant information that needs to be sent to the server, the medical device shall also pulse a key to the patient data hub; this key, once received by the patient data hub, will be matched to determine whether the two devices are in accordance.

Exemplary functional specifications would be as follows: Latency of communication <5 s. The side-band authentication provides an added layer of security. The side-band authentication feature includes two channels to increase security by conducting a secondary authentication on all data transferred from a medical device 18 to the communication device 10. In exemplary embodiments, the first and main channel is Bluetooth, and it may be used to transfer encrypted medical data from medical instruments 18 to communication device 10. The second channel, which could be ZigBee, is a separate low-power transceiver circuit that will be used to transmit a secondary authentication.

Bluetooth provides the security benefit of locking the signal down to a specific space. In the medical industry, this equates to confining the signal to a single hospital room, allowing the communication device 10 to easily communicate with the necessary medical instruments at the patient's bedside while preventing nonlocal attacks. In exemplary embodiments, some or all devices will move to dual communication channels. The main data channel could be Bluetooth and will transfer all medical data to the communication device 10, and the second channel could be used for local authentication. In exemplary embodiments, any medical data transferred to the hospital server may undergo two different authentication processes, so all data has been duly authenticated.

It should be noted that the next generation of medical devices could communicate through disclosed systems and methods, and by taking the medical devices off of the hospital network, the security is greatly increased. This is achieved as the communication device 10 acts as a buffer to any Denial of Service attacks to medical instruments. While theoretically the communication device 10 is still susceptible, only the information transfer will be inhibited until the attack is resolved, not the devices that patients' lives depend on.

While the disclosed systems and devices have been described in terms of what are presently considered to be the most practical exemplary embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

Thus, it is seen that improved communication devices, systems, and methods are provided. It should be understood that any of the foregoing configurations and specialized components may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

1. A communication device comprising:
a housing;
a transceiver circuit disposed within the housing, the transceiver circuit having at a communication channel;
a processor in communication with the transceiver circuit;
a battery circuit disposed within the housing;
wherein the communication channel receives medical data from at least one medical instrument.

2. The communication device of claim 1 wherein the transceiver circuit transmits the medical data to a network or server.

3. The communication device of claim 1 wherein the at least one medical instrument is located in a room and the transceiver circuit does not receive medical data when located outside the room.

4. The communication device of claim 1 wherein the processor develops a health status proxy based upon the medical data.

5. The communication device of claim 1 wherein the medical data is received, authenticated, and transmitted in real time.

6. The communication device of claim 2 wherein the network or server contains a patient data hub and the transceiver transmits a key to the patient data hub.

7. The communication device of claim 1 further comprising a user interface displaying medical information of a patient.

8. The communication device of claim 7 further comprising a warning system alerting a medical practitioner that a patient requires medical attention.

9. The communication device of claim 7 wherein the user interface enables a medical practitioner to set medical information parameters such that the communication device will monitor for deviations from the set medical information parameters.

10. A computer-implemented system of analyzing, authenticating, and transmitting medical information, comprising:
one or more medical instruments;
a server or network; and
a communication device in communication with the one or more medical instruments and the server or network, the communication device including
a housing;
a transceiver circuit disposed within the housing, the transceiver circuit having at a communication channel;
a processor in communication with the transceiver circuit; and
a battery circuit disposed within the housing;
wherein the communication channel receives medical data from the one or more medical instruments; and
wherein the transceiver circuit transmits the medical data to a network or server.

11. The system of claim 10 wherein the network or server comprises a port that automatically opens such that the network or server listens for and connects to a first medical instrument of the one or more medical instruments.

12. The system of claim 11 wherein after the network or server completes a connection to the first medical instrument of the one or more medical instruments, the network or server automatically listens for and connects to a second medical instrument of the one or more medical instruments.

13. The system of claim 10 wherein the network or server contains a patient data hub and the transceiver transmits a key to the patient data hub.

14. The system of claim 10 wherein the one or more medical instruments comprise a plurality of medical instruments connected to a single patient.

15. The system of claim 10 further comprising a warning system alerting a medical practitioner that a patient requires medical attention.

16. A computer-implemented method of analyzing, authenticating, and transmitting medical information, comprising:
receiving medical data from one or more medical instruments;
authenticating the medical data;
transmitting the medical data to a network or server;
developing a health status proxy based upon the medical data;
wherein the receiving, authenticating, and transmitting is performed in real time.

17. The method of claim 16 wherein the network or server contains a patient data hub and further comprising transmitting a key to the patient data hub.

18. The method of claim 16 further automatically listening for and connecting to a communication device that performs the receiving, authenticating, and transmitting steps.

19. The method of claim 16 further comprising automatically listening for and connecting to a first medical instrument of the one or more medical instruments.

20. The method of claim 16 further comprising alerting a medical practitioner that a patient requires medical attention.
